(19) United States
(12) Patent Application Publication
(10) Pub. No.: US 2025/0360237 A1
(43) Pub. Date: Nov. 27, 2025

(54) TARGETING SYSTEM FOR CANCER TREATMENT

(71) Applicant: Coretag IP B.V., Rotterdam (NL)

(72) Inventor: Markwin Hendrik MARING, Rotterdam (NL)

(73) Assignee: CORETAG IP B.V., Rotterdam (NL)

(21) Appl. No.: 19/128,946

(22) PCT Filed: Nov. 9, 2023
(86) PCT No.: PCT/NL2023/050592

§ 371 Date: May 9, 2025

(30) Foreign Application Priority Data
Nov. 9, 2022 (NL) 2033489

(51) Int. Cl. A61K 51/04; A61P 35/00

(52) U.S. Cl. CPC A61K 51/0497; A61K 51/0446; A61P 35/00; A61K 2121/00

(57) ABSTRACT

The invention relates to a targeting system for cancer comprising a cancer treatment moiety, such as radioisotope, with improved therapeutic effect, the use thereof as a medicament, such as for cancers, to a dosage comprising the targeting system, the use thereof in therapy, and the use thereof in treatment, as well as to a method of forming a targeting system. In particular the invention relates to systems targeting cancer cells.

### FIELD OF THE INVENTION

The invention relates to a targeting system for cancer comprising a cancer treatment moiety, such as radioisotope, with improved therapeutic effect, the use thereof as a medicament, such as for cancers, to a dosage comprising the targeting system, the use thereof in therapy, and the use thereof in treatment, as well as to a method of forming a targeting system. In particular the invention relates to systems targeting cancer cells.

### BACKGROUND OF THE INVENTION

The present invention relates to targeting of cancer cells with a targeting system, which is administered to a human or animal body. This is an alternative to radiation therapy or other forms of cancer therapy. The targeting system comprises a cancer treatment moiety, such as a radioisotope. It has been found that targeting systems are rather prone to degradation, in particular degradation after such a targeting system reaches an intended location, i.e. the cancer. Therefore, such systems are not as effective as expected.

The present invention is amongst others in the field of radiation therapy. Radiation therapy uses ionizing radiation, generally provided as part of cancer treatment to control, or kill malignant cells. Radiation therapy may be if they are administered localized to one area of the body. It may also be used to prevent tumour recurrence. Radiation therapy may be used in combination with chemotherapy. Radiation therapy is typically applied to the cancerous tumour cells as it limits cell growth thereof. Radiation oncology is the medical specialty concerned with prescribing radiation, and is distinct from radiology, the use of radiation in medical imaging and diagnosis. Most common cancer types can be treated with radiation therapy in some way. The precise treatment intent may depend on the tumour type, location, and stage, as well as the general health of the patient.

Various prior art documents may be referred to relating to recent developments and issues still being present in radiotherapy. WO 2019/115684 A1 recites complexes comprising a prostate-specific membrane antigen (PSMA) targeting compound linked to a radionuclide, such as ²¹²Pb or ²²⁷Th, through a TCMC or DOTA chelating moiety. These compounds, and pharmaceutical compositions comprising them, can be used for medical applications. These applications include the treatment of prostate cancer, and the complexes allow for dual targeting of cancers. WO 2018/132751 A1 recites a cancer targeting composition, kit, and method for treatment of cancer cells overexpressing somatostatin receptors is disclosed. The composition includes a radioisotope, a chelator, and a targeting moiety. The chelator includes a nitrogen ring structure including a tetra-azacyclododecane, a tri-azacyclononane, and/or a tetra-azabicyclo [6.6.2] hexadecane derivative. The targeting moiety includes a somatostatin receptor targeting peptide. The somatostatin receptor targeting peptide includes an octreotide derivative. The targeting moiety is chelated to the radioisotope by the chelator whereby the cancer cells are targeted for elimination. WO 2022/180126 A1 recites a targeting system with improved uptake, the use thereof as a medicament, such as for cancers, to a dosage comprising the targeting system, the use there-of in therapy, the use thereof in treatment, and the use thereof in diagnosis or imaging. In particular the invention relates to systems targeting necrotic cells. An article by Li et al. (doi:10.1016/J.APRADISO.2017.05.006) recite a method for preparation of Pb²¹² and Ph²⁰³ labelled chelator-modified peptide-based radiopharmaceuticals for cancer imaging and radionuclide therapy has been developed and adapted for automated clinical production. Pre-concentration and isolation of radioactive Pb²⁺ from interfering metals in dilute hydrochloric acid was optimized using a commercially-available Pb-specific chromatography resin packed in disposable plastic columns. The pre-concentrated radioactive Pb²⁺ is eluted in NaOAc buffer directly to the reaction vessel containing chelator-modified peptides. Radiolabelling was found to proceed efficiently at 85° C. (45 min; pH 5.5). The specific activity of radiolabelled conjugates was optimized by separation of radiolabelled conjugates from unlabelled peptide via HPLC. US 2019/321495 A1 recites compositions, kits and methods to treat a hyperproliferative disorder with an agent that increases expression of MCR1 and an MCR1 ligand. The invention also provides a method of treating drug-resistant melanoma, comprising administering an MCR1 ligand to a patient in need thereof. The invention also provides in certain embodiments a melanoma-targeting conjugate comprising Formula (I): T-L-X wherein T is a MCR1 ligand, L is a linker, and X an anti-cancer composition, for the therapeutic treatment of a hyperproliferative disorder. Sathekge et. al (doi:10.1007/s00259-017-3657-9) recites ²¹³Bi-PSMA-617 targeted alpha-radionuclide therapy in metastatic castration-resistant prostate cancer in a patient with mCRPC that was progressive under conventional therapy. The patient was treated with two cycles of ²¹³Bi-PSMA-617 with a cumulative activity of 592 MBq. Restaging with ⁶⁸Ga-PSMA PET/CT after 11 months showed a remarkable molecular imaging response. This patient also demonstrated a biochemical response (decrease in PSA level from 237 μg/L to 43 μg/L). Kostelnik et al. (doi:10.1021/acs.chemrev.8b00294) recite fundamental concepts of drug design and applications, with particular emphasis on bifunctional chelators (BFCs), which ensure secure consolidation of the radiometal and targeting vector and are integral for optimal drug performance. Also presented are detailed accounts of production, chelation chemistry, and biological use of selected main group and rare earth radiometals. Ahenkorah et al. (doi:10.3390/pharmaceutics13050599) recite radionuclide properties and production of ²²⁵Ac and its daughter ²¹³Bi are discussed, followed by the fundamental chemical properties of bismuth. Next, an overview of available acyclic and macrocyclic bifunctional chelators for bismuth and general considerations for designing a ²¹³Bi-radiopharmaceutical are provided. Finally, we provide an overview of preclinical and clinical studies involving ²¹³Bi-radiopharmaceuticals, as well as the future perspectives of this promising cancer treatment option. Dadachova et al. (DOI:10.1053/j.semnuclmed.2010.01.002) recite that actively targeted alpha-particles offer specific tumour cell killing action with less collateral damage to surrounding normal tissues than beta-emitters. Radiolabelled peptides that bind to different receptors on the tumours have been investigated as potential therapeutic agents both in the preclinical and clinical settings. Advantages of radiolabelled peptides over antibodies in- clude relatively straightforward chemical synthesis, versatility, easier radiolabelling, rapid clearance from the circulation, faster penetration and more uniform distribution into tissues, and less immunogenicity. Rapid internalization of the radiolabelled peptides with equally rapid re-expression of the cell surface target is a highly desirable property that enhances the total delivery of these radionuclides into malignant sites. Peptides, such as octreotide, alpha-melanocyte-stimulating hormone analogues, arginine-glycine-aspartic acid-containing peptides, bombesin derivatives, and others may all be feasible for use with alpha-emitters. Obstacles that continue to obstruct widespread acceptance of alpha-emitter-labelled peptides are primarily the supply of these radionuclides and concerns about potential kidney toxicity. New sources and methods for production of these medically valuable radionuclides and better understanding of mechanisms related to the peptide renal uptake and clearance should speed up the introduction of alpha-emitter-labelled peptides into the clinic. Garashchenko et al. (doi:10.1134/S1063778818100071) recite problems of the development of radiopharmaceuticals (RPs) based on alpha emitting radionuclides are discussed. The prospects of application of the radionuclides ²²⁷Th, ²²⁵Ac, ²²³Ra, ²¹³Bi, ²¹²Pb/²¹²Bi, ²¹²Bi, ²¹¹At, and ¹⁴⁹Th are estimated in the aspect of their physicochemical properties, such as half-life, properties of daughter radionuclides, and complexing ability. The methods used for the production of radionuclides and their industrial availability are considered. Some examples of radionuclide complexes with ligands and nanoparticles for targeted delivery are presented. The results of medical trials for RPs based on alpha emitters are given. And, and an article by Hossein et al. (doi:10.1007/S00259-021-05405-0) recite novel developments in radiochemistry, and availability of relevant a-emitters for targeted therapy have provided innovative approaches to precision cancer management. The approval of ²²³Ra dichloride for treatment of men with osseous metastatic castrate-resistant prostate cancer unleashed targeted a-therapy as a safe and effective cancer management strategy. While there is currently active research on new α-therapy regimens for prostate cancer based on the prostate-specific membrane antigen, there is emerging development of radiopharmaceutical therapy with a range of biological targets and α-emitting radioisotopes for malignancies other than the prostate cancer. This article provides a brief review of preclinical and first-in-human studies of targeted α-therapy in the cancers of brain, breast, lung, gastrointestinal, pancreas, ovary, and the urinary bladder. The data on leukemia, melanoma, myeloma, and neuroendocrine tumours will also be presented. It is anticipated that with further research the emerging role of targeted α-therapy in cancer management will be defined and validated.

It is an object of the present invention to overcome one or more disadvantages of the targeting systems of the prior art and to provide alternatives to current systems for treatment of cancers, without jeopardizing functionality and advantages.

### SUMMARY OF THE INVENTION

It has now been found that cancer treatment can be improved by providing a targeting system, in particular enhanced uptake of cancer treatment moieties in a tumour, improved cancer treatment moiety half-life time, whereas also improved retention and permeation are expected, an albumin binding moiety (AB), attached to the albumin binding moiety a targeting molecule (TM), wherein the targeting molecule is a cyanine, that is capable of interacting with a necrotic cell of proteins thereof, and wherein the albumin binding moiety comprises an carboxylic acid residue moiety, the carboxylic acid moiety attached to a phenyl moiety, and attached to the phenyl moiety at least one first chemical moiety, wherein the at least one first chemical moiety is selected from F, phenyl, C, O, CF₃, OCH₃, NO₂, NH₂, CH₃, Cl, OCF₃, and combinations thereof, and attached to the targeting molecule a cancer treatment moiety. It is noted that in general cancer can be treated by surgery, chemotherapy, radiation therapy, hormonal therapy, targeted therapy (including immunotherapy such as monoclonal antibody therapy) and synthetic lethality, most commonly as a series of separate treatments (e.g. chemotherapy before surgery). The present invention in particular relates to a combination of chemotherapy, in particular by using a radionuclide therein, and targeted therapy. The present cancer treatment is, as it already indicates, effective in the treatment of cancer. The moieties and likewise molecules of the present targeting system may be bound directly to one and another, or may have intermediate molecules between them, or a combination thereof. It is found that by providing such a specific targeting system, the targeting system is much more stable, and actually reaches the intended location in the body. The present invention therefore relates to a targeting system which is much more effective than prior art targeting system, and in particular causes less side effects. Prior art targeting systems and the present system typically arrive at an intended location. However, prior art targeting systems, and in particular those with radioisotopes that emit more than one alfa-particle, cause damage to human/animal tissue and bones elsewhere by emitting the further alfa-particles. The present targeting system has typically much less side effects, and often not noticeable side effects. In a first aspect the present invention relates to a targeting system comprising a targeting molecule for binding to cells, such that the targeting system arrives at the intended location, the targeting molecule being selected from cyanines, wherein the targeting molecule may be attached to a linker, wherein the linker may be attached to a radio-isotope or a radioisotope binding molecule, and wherein the radio-isotope may be an alfa-emitter capable of directly or indirectly emitting only one alfa particle upon decay of the radio-isotope, wherein a half-life of the radio-isotope is >0.5 hours and <1000 days, in particular >1 hour, more in particular >7 hours. In the exemplary embodiment, with directly or indirectly emitting only one alfa particle upon decay of the radio-isotope it is meant that the radioisotope, upon decay, and optional further decay, as may be the case with radioisotopes, emits in the full chain of decay only one, that is a single, alfa-particle. The present radioisotope is not a multiple alfa-emitter, such as ²³²Th, which produces 6 alfa-particles, in addition to 4 beta-particles (upon reaching a stable isotope), or ²³³U, which produces 7 alfa-particles, in addition to 3 beta-particles. The half-life of the present radioisotope, in view of the alfa-particle being emitted, is not too short, as in that case it (largely) decays before application, and not too long, as in that case limited radio-activity and therefore therapy is obtained. So it is found that commonly used alfa-emitters like ²²⁵Ac have a fairly long half-life of 10 days, so they need to be in the right location for a longer period of time to do their job properly. The Ac²²⁵ may actually not slowly disappear from the tumour tissue, because it will then cause damage in other places. The relatively long half-life of 10 days is then a problem. If it circulates in the bloodstream for a long time, it can get everywhere and cause damage to healthy tissue. If the Ac²²⁵ stays in the right place, the first alpha decay is then “on target”: the alpha particle does the damage to the tissue or the like in the right place. The decay changes the Actinium²²⁵ into another unstable element, which again emits an alpha, etc.: in total, the Ac²²⁵ eventually changes into stable Bismuth²⁰⁹, emitting four alpha particles along the way. During the first decay, the Ac²²⁵ (which has then become Fr²²¹) shoots away. It can move freely. Also in the decay chain is Bismuth²¹³ which gives off a gamma that can be measured well with a scanner: so in this way one can determine where the elements in the decay chain of Ac²²⁵ remain in the patient. It is found that Bi²¹³ is widely distributed (i.e. does not remain localized in tumour tissue) through the human or animal body. As a consequence alpha damage will also occur in healthy tissue. These side effects are not really seen or recorded now: the patients, especially in Germany, are treated on an individual and commercial basis, with no group study or tracking system, and they are mostly late-stage patients who have died before side damage is expressed. Ac²²⁵ could do its job rather perfect, if the targeting is right, and the Ac²²⁵ remains in the right place throughout the full decay chain. But then a lot of conditions have to be met that are not just trivial. This is typically not achieved in practice. On the other hand the present single alpha emitter with a relatively short half-life releases 1 alpha in said short time: and the collateral damage with good targeting is limited. Pb²¹² is such an isotope: it is not an alpha emitter itself, but a beta emitter (emits an electron, just like Lutetium), with 1 alpha emission in the decay chain, until it is stable (it then becomes Pb²⁰⁸). In general, beta emission will not cause the atom to break free: thus, it stays in place and the alpha is then on target.

The present targeting system comprises at least three entities, and typically four to six entities, the entities being joined or linked, such as by a chemical or physical bond, each entity serving a distinct function within the system.

The present targeting molecule may be attached to a further entity, namely a linker, the linker being attached to the present radioisotope, which latter molecule may be attached to a chelator, wherein the chelator is typically selected from DOTA, C-DOTA, NOTA and TCMC-comprising compounds (DOTA (CAS Number 60239-18-1): 1,4,7,10-Tetraazacyclododecane-1,4,7,10-tetraacetic acid, TCMC (CAS Number: 2153478-57-8): 2-[(4-Isothiocyanatophenyl)methyl]-1,4,7,10-tetraazacyclododecane-1,4,7,10-tetraacetamide tetrahydrochloride; and NOTA (CAS Number, 56491-86-2): 1,4,7-triazacyclononane-N,N′,N″-triacetic acid). The targeting molecule is for binding to cells, typically to receptors thereof. Thereto anti-bodies, peptides, small targeting molecules, and kinase inhibitors, may be used. The linker may be any suitable linker. The present system is found to be effective as a medicament, also referred to as drug, such as for cancers, etc.

In a second aspect the present invention relates to a dosage, for use as a medicament, typically for use in therapy, in particular in radio-therapy, or for use in treatment.

In a further aspect the present invention relates to a use of a dosage according to the invention in particular for use as a medicament, such as a drug for treatment of cancers selected from breast, kidney, non-Hodgkin's lymphoma, prostate, bladder, esophagus, pharynx and larynx, lung, brain, pancreas, colorectal, head neck, glioblastoma, myeloma, myometrium, ovarian, gastrointestinal stromal cancer, tumours thereof, or metastases thereof, comprising an effective amount of the present targeting system.

Advantages of the present description are detailed throughout the description.

### DETAILED DESCRIPTION

It is noted that examples given, as well as embodiments are not considered to be limiting. The scope of the invention is defined by the claims.

In an exemplary embodiment of the present targeting system the carboxylic acid moiety is attached to the phenyl moiety by a C₁-C₆ moiety, in particular by a C₂-C₃ moiety, optionally comprising a ketone or consisting of a ketone.

In an exemplary embodiment of the present targeting system. the at least one first chemical moiety is selected from H, F, phenyl, C, O, I, CF₃, F, OCH₃, NO₂, NH₂, CH₃, Cl, OCF₃, and combinations thereof, in particular from CF₃, phenyl, I, H, F, and combinations thereof, more in particular from

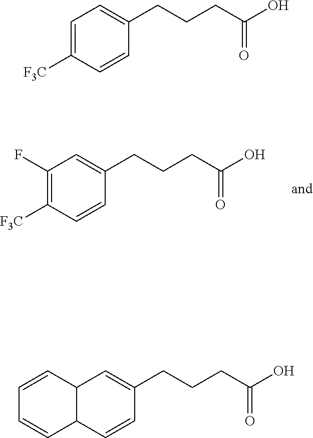 [SMILES: O=C(O)CCCc1ccc(C(F)(F)F)cc1 ; O=C(O)CCCc1ccc(C(F)(F)F)c(F)c1 ; O=C(O)CCCC1=CC2C=CC=CC2C=C1]

These chemical moieties are found to provide significant tumour uptake increase, compared to cancer treatment moieties alone, good in vivo performance, good (limited) biodistribution, and good tumour/non-tumour ratios.

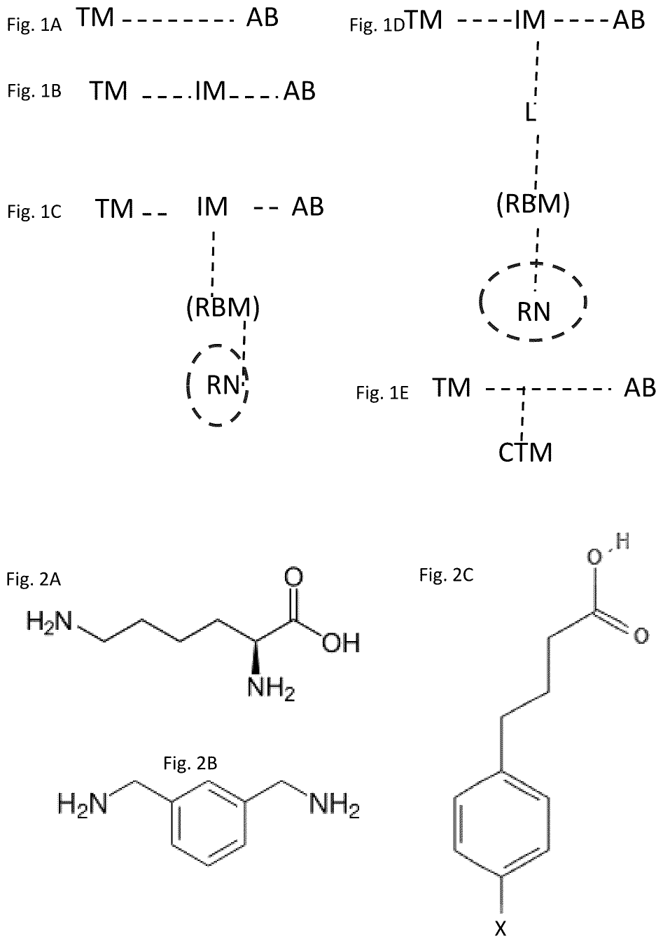

In an exemplary embodiment of the present targeting system the cyanine is selected from Streptocyanines, hemicyanines, closed cyanines, neutrocyanines, merocyanines, azacyanines, and apocyanines, in particular from a non-reactive cyanine dye, according to FIG. 1 I, II and III,

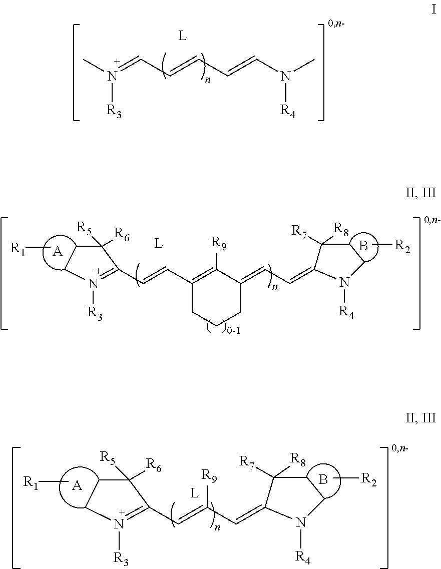

wherein n is an integer, such as n ∈[2,10], in particular n ∈[4,8], the chain L has up to n-1 double bonds, in particular n/2 double bonds,
wherein sub-families II and III may comprise respectively one and two aromatic ring systems (A,B) signified by the curved line(s) C,
wherein A,B are preferably selected each individually from benzene and naphthalene,
wherein further groups R5, R6, R7, and R8, may be present, R5, R6, R7, and R8, are preferably selected each individually from H, and alkyl, such as methyl, ethyl, and propyl, preferably methyl,
wherein the aromatic ring systems may comprise further functional groups R1, R2, and/or substituents, R1, R2, are preferably selected each individually from H, sulphonate, and sulphonamide,
wherein the chain of alternating single and double bonds L may be interrupted by one or more partly and fully saturated ring structures, such as cyclopentene and cyclohexene, and combinations thereof, such as one or more cyclohexene rings, wherein the saturated ring structure may further comprise functional groups R9, being selected from R10, H, AA and BB, wherein R10 is selected from, H, SO₃H, Cl, —N—C═O—(CH₂)_{q—Y}3 (q=1-6), —(CH₂)ᵣ—Y4 (r=1-6), Y3 and Y4 are each independently one of H, COOH, SO₃H, and CN,
wherein the nitrogen atoms (N) may comprise further functional N-side groups R3, R4,
wherein R3, R4 are preferably selected each individually from —(CH₂)ₘY, wherein Y is selected each individually from a carboxylic acid having 1-4 carbon atoms, a sulphonate group, CN, C≡C, and C═C, and salts thereof,
wherein said N-side groups comprise m carbon atoms, such as m ∈[1,10], preferably m ∈[2,8], more preferably m ∈[3,7], most preferably m=4,5, and 6,
even more preferably at least one of m=4, 5, and 6, preferably one m=6, and the other m preferably is 4, 5 or 6,
wherein said N-side groups comprise one or more functional groups on an end opposing the N, such as a carboxylic acid having 1-4 carbon atoms, an sulphonic group, and salts there-of, such as sodium and potassium salts, most preferably the functional group on the end comprises one or more double C—C bonds, preferably a carboxylate thereof, and/or
wherein the targeting molecule is neutral or negatively charged,
more in particular CW-800, 800RS and ZW-800, and combinations thereof,
in particular wherein the cyanine comprises at least one SO₃H moiety, more in particular 2-4 SO₃H moieties. These cyanines are found to have very good targeting characteristics.

In an exemplary embodiment of the present targeting system the albumin binding moiety (AB) is attached to the targeting molecule (TM) by an intermediate moiety (IM), that is, a chemical moiety in between and attached to the albumin binding moiety and the targeting molecule, in particular wherein the intermediate moiety (IM) is selected from amino acid comprising residues, wherein the amino acid residue is preferably selected from Alanine, Arginine, Asparagine, Aspartic acid, Cysteine, phenylalanine, Glutamine, Glutamine acid, Glycine, Histidine, Isoleucine, Leucine, Lysine, Methionine, Proline, Pyrroline, Selenocysteine, Serine, Threonine, Tryptophan, Tyrosine, and Valine, in particular Lysine, such as L-Lys.

In an exemplary embodiment of the present targeting system the cancer treatment moiety comprises a radionuclide (RN), wherein the radionuclide is attached to the intermediate moiety (IM), in particular wherein the radionuclide is attached to a radionuclide binding molecule (RBM), which radionuclide binding molecule (RBM) is attached to the intermediate moiety (IM). Therewith a stable complex is formed.

In an exemplary embodiment of the present targeting system the radionuclide binding molecule (RBM) is attached to a linker (L), in particular wherein the linker is attached to the intermediate moiety (IM).

In an exemplary embodiment of the present targeting system the radio-isotope is selected from ²¹¹At (7.2 hours), ²¹⁰Bi (5.0 days), ²¹²Bi (60,5 min), ²¹³Bi (45 min), ²¹²Pb (10 hours), ²¹⁰Po (138 days), and ¹⁴⁹Tb (4 hours), and combinations thereof. It is found that by providing such a specific radioisotope, which in decay provides a single alfa, and possibly a beta or gamma particle in addition to the single alfa-particle, the targeting system is much more stable, and actually reaches the intended location in the body.

In an exemplary embodiment of the present targeting system the radionuclide, also referred to as radioisotope, is present as a cation, such as with a valence of 0, 1, 2, 3, or 4.

In an exemplary embodiment of the present targeting system upon decay of the single alfa particle >3 MeV energy is released, in particular >5 MeV, such as >6MeV.

In an exemplary embodiment the present targeting system further comprises at least one radio-isotope binding molecule, wherein the binding molecule is attached to the linker and the radio-isotope, in particular wherein the binding molecule is a chelator, such as DOTA, NOTA, CDOTA, TCMC, and DOTA and NOTA comprising compounds (DOTA: 1,4,7,10-Tetraazacyclododecane-1,4,7,10-tetraacetic acid and NOTA: 1,4,7-triazacyclononane-N,N′,N″-triacetic acid).

In an exemplary embodiment of the present targeting system the linker is selected from poly(ethylene)glycol (PEG) linkers, in particular wherein the linker is selected from PEG-linkers with n in H—[O—CH₂—CH₂]ₙ—OH from 3-30, more preferably with n from 4-6, and from moieties comprising at least two functional groups selected from OH, NH₂, and COOH, in particular from aromatic moieties, more in particular from diamines, even more in particular from phenyl diamines and naphthalene diamines.

In an exemplary embodiment of the present targeting system the targeting molecule is neutral or negatively charged.

In an exemplary embodiment of the present targeting system the radionuclide binding molecule is selected from TCMC, 2,2′,2″,2′″-(1,4,7,10-tetraazacyclododecane-1,4,7,10-tetrayl)tetra acetic acid (DOTA), Hexahydro-1H-1,4,7-triazonine-1,4,7-triacetic acid (NOTA), 1,4,7-Tris(phosphonomethyl)-1,4,7-triazacyclononane (NOTP), ((1,4,7-triazonane -1,4,7-triyl)tris(methylene))tris(phosphinic acid)(TRAP), N′-[5-[[4-[[5-(acetylhydroxyamino)pentyl]amino]-1,4-dioxobutyl]hydroxyamino]pentyl]-N-(5-aminopentyl)-N-hydroxy -butanediamide (DFO), 2,2′,2″,2′″-((((carboxymethyl)azanediyl)bis(ethane-2,1- diyl))bis(azanetriyl))tetraacetic acid (DTPA), 3,12-bis(carboxymethyl)-6,9-dioxa-3,12-diazatetradecanedioic acid (EGTA), 2,2′,2″,2′″-(ethane-1,2-diylbis(azanetriyl))tetraacetic acid (EDTA), 7-[2-[bis(carboxymethyl)amino]-3-(4-nitrophenyl)propyl]hexahydro-1H-1,4,7-Triazonine-1,4(5H)-diacetic acid (C-NETA), 2-(4,7-bis(carboxymethyl)-1,4,7-triazonan-1-yl)pentanedioic acid (NODAGA), 2-(4,7, 10-tris(carboxymethyl)-1,4,7, 10-tetraazacyclododecan-1-yl)-pentanedioic acid (DOTAGA), 1,4,7-triazacydononane-1-[methyl (2-carboxyethyl)-phosphinic acid]-4,7-bis[methyl(2-hydroxymethyl)phosphinic acid] (NOPO), 3,6,9, 1 5-tetraazabicyclo[9,3, 1 ]pentadeca-1 (15), 11, 13-triene-3,6,9-triacetic acid (PCTA), N,N″-bis[2-hydroxy-5-(carboxyethyl)-benzyl]ethylenediamine-N,N″-diacetic acid (HBED-CC), N,N′-bis(2,2-dimethyl-2-mercaptoethyl)ethylenediamine-N,N′-diacetic acid (6SS), 1-(4-carboxymethoxybenzyl)-N-N′-bis[(2-mercapto-2,2-dimethyl)ethyl]-1,2-ethylenediamine-N,N′-diacetic acid (B6SS), N,N′-dipyridoxylethylenediamine-N,N′-diacetic acid (PLED), 1,1,1-Tris-(aminomethyl)ethane (TAME), nitrilotrimethylphosphonic acid (NTP), 2-BAPEN, 2,2′,2″,2′″-(1,4,8, 11-tetraazacyclotetradecane-1,4,8,11-tetrayl)tetraacetic acid (TOTA), and compounds comprising one of these radionuclide binding molecules.

In an exemplary embodiment the present targeting system is for use as a medicament for the treatment of a cancer, such as Acute Leukemia, AML, anaplastic large cell lymphoma, neuroblastoma, bladder cancer, bone marrow, brain, breast and ovarian cancer, colorectal, urothelial carcinomas, cholangiocarcinoma, chronic lymphocytic leukaemia, non-Hodgkin lymphoma, non-Hodgkin's disease, distant colorectal cancer, GEP-NET, glioma, glioblastoma, colorectal, lung, oesophageal and stomach cancer, head & neck carcinoma, haematological cancers, HER2 positive breast cancer, HR-Pos and HER2-negative breast cancer, immuno oncology, late-stage melanoma, leukaemia, lung & breast Cancers, lymphoma, medullary thyroid cancer, NSCLC, SCLC, melanoma, metastatic breast cancer, metastatic colorectal cancer, advanced GIST, metastatic mCR-prostate cancer (bone), metastatic melanoma, myeloma, multi cancers, myeloid leukaemia, myeloid leukaemia, Philadelphia chromosome positive acute lymphoblastic leukaemia, neuroblastoma, neuroendocrine tumours, small cell lung cancer, Non-small cell lung cancer, small cell lung cancer, non-Hodgkin lymphoma, Parkinson disease, primary Kidney Cancer, Advanced renal cancer, advanced primary liver cancer, FLT3-ITD passive AML and radioactive iodine resistant advanced thyroid carcinoma), prostate cancer, renal cell carcinoma (RCC), Imatinib-resistant GIST, Renal cell carcinoma, soft tissue sarcoma, rheumatoid arthritis, psoriatic arthritis, and ulcerative colitis, Targeted delivery of vinblastine, and Thyroid cancer.

In an exemplary embodiment the present dosage is for use as a medicament, such as a drug for treatment of cancers selected from breast, kidney, non-Hodgkin's lymphoma, prostate, bladder, esophagus, pharynx and larynx, lung, brain, pancreas, colorectal, head neck, glioblastoma, myeloma, myometrium, ovarian, gastrointestinal stromal cancer, tumours thereof, or metastases thereof, comprising an effective amount of the targeting system of the invention.

In an exemplary embodiment the present dosage comprises an amount of 0.1-1000 nMole targeting system/kg body weight and/or is provided in a physiological acceptable solution of 1-50 ml.

The present invention also relates to multiple dosages according to the invention, such as 2-4 dosages, for intermittent application, such as with intervals of 0.2-4 hours, preferably 1-2 hours.

And further to a method of establishing a dosage, comprising determining a body weight in kg, and multiplying the body weight with 0.1-1000 nMole targeting system according to the invention.

In an exemplary embodiment of the present targeting system the linker is selected from poly (ethylene) glycol (PEG) linkers, preferably PEG-linkers with n in H—[O—CH₂—CH₂]ₙ—OH from 3-30, more preferably with n from 4-6.

In an exemplary embodiment the present targeting system is for use as a medicament for the treatment of a cancer, such as Acute Leukemia, AML, anaplastic large cell lymphoma, neuroblastoma, Bladder Cancer, Bone marrow, brain, Breast and Ovarian Cancer, Colorectal, Urothelial Carcinomas, Cholangiocarcinoma, Chronic lymphocytic leukaemia, non-Hodgkin lymphoma, Distant colorectal cancer, GEP-NET, Glioma, colorectal, lung, oesophageal and stomach cancer, Head & Neck Carcinoma, Haematological Cancers, HER2 positive breast cancer, HR-Pos and HER2-negative breast cancer, Immuno Oncology, Late-stage melanoma, Leukemia, Lung & Breast Cancers, Lymphoma, Medullary Thyroid Cancer, NSCLC Melanoma, Metastatic breast cancer, Metastatic Colorectal Cancer, Advanced GIST, metastatic mCR-Prostate Cancer (bone), Metastatic Melanoma, Multi cancers, Myeloid Leukemia, Myeloid Leukemia, Philadelphia Chromosome Positive Acute lymphoblastic leukaemia, Neuroblastoma, Neuroendocrine Tumours, Non-Small Cell Lung Cancer, Non-Hodgkin lymphoma, Parkinson Disease, Primary Kidney cancer, Advanced renal cancer, advanced primary liver cancer, FLT3-ITD positive AML and radioactive iodine resistant advanced thyroid carcinoma), Prostate Cancer, Renal Cell Carcinoma (RCC), Imatinib-resistant GIST, Renal cell carcinoma, soft tissue sarcoma, rheumatoid arthritis, psoriatic arthritis, and ulcerative colitis, Targeted delivery of vinblastine, and Thyroid cancer.

In a further aspect the present invention relates to a dosage for use as a medicament, such as a drug for treatment of cancers selected from esophagus, pharynx and larynx, lung, brain, Pancreas, colorectal, head neck, glioblastoma, myometrium, ovarium, Gastrointestinal stromal cancer, tumours thereof, or metastases thereof, comprising an effective amount of the present targeting system.

In an example the dosage comprises an amount of 0.1-1000 nMole targeting system/kg body weight, preferably 0.5-500 nMole targeting system/kg body weight, more preferably 1-250 nMole targeting system/kg body weight, even more preferably 2-100 nMole targeting system/kg body weight, such as 5-50 nMole targeting system/kg body weight; such may relate to a dosage of e.g. 0.01-200 mgram. The dosage preferably is provided in a physiological acceptable solution of 1-50 ml. Preferably a kit comprising some (1-50) dosages is provided.

The invention is further detailed by the Examples and accompanying figures, which are exemplary and explanatory of nature and are not limiting the scope of the invention. To the person skilled in the art it may be clear that many variants, being obvious or not, may be conceivable falling within the scope of protection, defined by the present claims.

### EXAMPLES/FIGURES

### Summary of the Figures

FIGS. 1a-e show exemplary targeting systems according to the invention.

FIGS. 2a-2c show exemplary moieties.

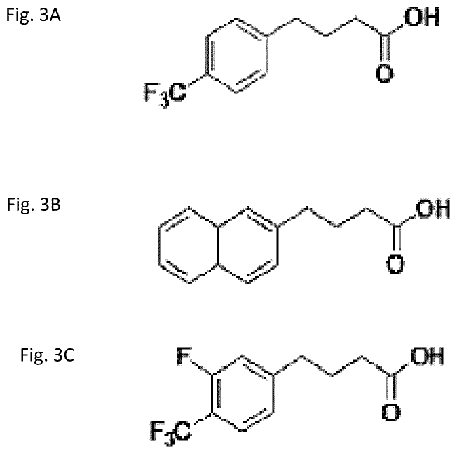

FIG. 3a-c show exemplary albumin binders.

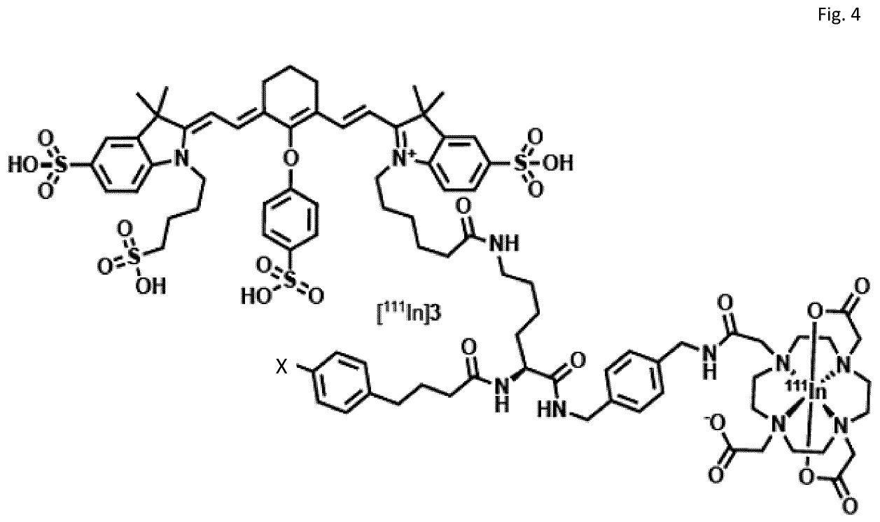

FIG. 4 shows an exemplary targeting system.

### DETAILED DECRIPTION OF THE FIGURES

FIG. 1a shows a schematical representation of the present an albumin binding moiety (AB) attached to the targeting molecule (TM). FIG. 1b shows a schematical representation of the present an albumin binding moiety (AB) attached to the targeting molecule (TM), with an intermediate molecule (IM). FIG. 1c shows a schematical representation of the present an albumin binding moiety (AB) attached to the targeting molecule (TM), with an intermediate molecule (IM), and attached the IM a radionuclide (RN). The radionuclide may be attached to the IM directly, or through an radionuclide binding molecule (RBM). FIG. 1d shows a schematical representation of the present an albumin binding moiety (AB) attached to the targeting molecule (TM), with an intermediate molecule (IM), and attached the IM a radionuclide (RN). The radionuclide may be attached to the IM directly, or through an radionuclide binding molecule (RBM), which RBM may be attached to the IM by a linker moiety. In FIGS. 1C-1D the cancer treatment moiety (CTM) is encircled, being or comprising in this case the RN. Fig. le shows the cancer treatment moiety (CTM) being attached to the targeting molecule.

FIG. 2a shows L-lys, FIG. 2b p-xylylene diamine, and FIG. 2c 4-(p-Xphenyl)-butyric acid. As alternatives to FIG. 2c the X may be F, Br, OCH₃, NH₂, NO₂, Cl, and CH₃.

FIG. 3a shows 4-(4-(trifluoromethyl)phenyl)butanoic acid, FIG. 3b shows 4-(3-fluoro-4-(trifluoromethyl)phenyl)butanoic acid, and FIG. 3c shows 4-naphthyl-butanoic acid, as exemplary albumin binders.

FIG. 4 shows an exemplary targeting system, namely 4-(p-X) butyramide-L-Lys(IRDye800CW)-p-xylylene diamine-DOTA, wherein X may be replaced by F, Br, OCH₃, NH₂, NO₂, Cl, and CH₃.

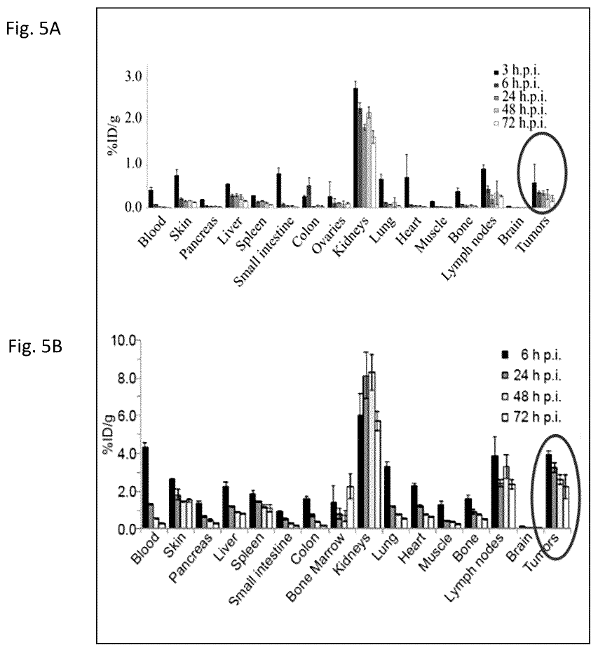

FIG. 5A show the comparative effect of ¹¹¹In-DOTO-PEG-800CW, with a tumour uptake of 0.3-0.5% ID/g, and FIG. 5B for ¹¹¹In-DOTO-PEG-800CW-IBA (X-phenyl butanoic acid), with a tumour uptake of 2-4% ID/g, clearly demonstrating the positive effect (4-10 times higher) of the present targeting molecule, in particular for blood, skin, pancreas, liver, spleen, small intestine, colon, ovaries, kidneys, lung, heart, muscle, bone, lymph nodes, brain, and averaged.

1. A targeting system for cancer treatment comprising an albumin binding moiety (AB), attached to the albumin binding moiety a targeting molecule (TM), wherein the targeting molecule is a cyanine, and wherein the albumin binding moiety comprises an carboxylic acid residue moiety, the carboxylic acid moiety attached to a phenyl moiety, and attached to the phenyl moiety at least one first chemical moiety, wherein the at least one first chemical moiety is selected from F, phenyl, C, O, CF₃, OCH₃, NO₂, NH₂, CH₃, Cl, OCF₃, and combinations thereof, and attached to the targeting molecule a cancer treatment moiety.

2. The targeting system according to claim 1, wherein the carboxylic acid moiety is attached to the phenyl moiety by a C₁-C₆ moiety.

3. The targeting system according to claim 1, wherein the cyanine is selected from Streptocyanines, hemicyanines, closed cyanines, neutrocyanines, merocyanines, azacyanines, and apocyanines, wherein the cyanine is a non-reactive cyanine dye, according to FIG. 1 I, II and III, 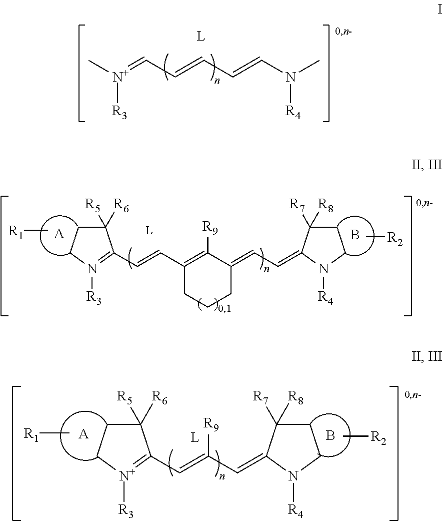
wherein n is an integer, the chain L has up to n-1 double bonds, wherein sub-families II and III comprise respectively one and two aromatic ring systems (A,B) signified by the curved line(s) C, wherein A,B are selected each individually from benzene and naphthalene.

4. The targeting system according to claims 1, wherein the albumin binding moiety (AB) is attached to the targeting molecule (TM) by an intermediate moiety (IM).

5. The targeting system according to claim 4, wherein the intermediate moiety (IM) is selected from amino acid comprising residues, wherein the amino acid residue is selected from Alanine, Arginine, Asparagine, Aspartic acid, Cysteine, phenylalanine, Glutamine, Glutamine acid, Glycine, Histidine, Isoleucine, Leucine, Lysine, Methionine, Proline, Pyrroline, Selenocysteine, Serine, Threonine, Tryptophan, Tyrosine, and Valine.

6. The targeting system according to claim 4, wherein the cancer treatment moiety comprises a radionuclide (RN), wherein the radionuclide is attached to the intermediate moiety (IM), wherein the radionuclide is attached to a radionuclide binding molecule (RBM), which radionuclide binding molecule (RBM) is attached to the intermediate moiety (IM).

7. The targeting system according to claim 6, wherein the radionuclide binding molecule (RBM) is attached to a linker (L), wherein the linker is attached to the intermediate moiety (IM).

8. The targeting system according to claim 6, wherein the radionuclide is present as a cation, with a valence of 0, 1, 2, 3, or 4, and/or wherein the radionuclide is selected from an alfa particle releasing radionuclide, and from a beta particle releasing radionuclide.

9. The targeting system according to claim 8, whereon upon alfa-decay of the radionuclide >3 MeV energy is released, and wherein the radio-nuclide is an alfa-emitter capable of directly or indirectly emitting only one alfa particle upon decay of the radio-nuclide, wherein a half-life of the radio-nuclide is >0.5 hours and <1000 days, wherein the radio-nuclide is selected from ¹¹¹In, ²¹¹At, ²¹⁰Bi, ²¹²Bi, ²¹²Pb, ²¹⁰Po, and ¹⁴⁹Tb, and combinations thereof.

10. The targeting system according to claim 7, wherein the linker is selected from poly(ethylene)glycol (PEG) linkers, wherein the linker is selected from PEG-linkers with n in H—[O—CH₂—CH₂]ₙ—OH with n from 3-30, and from moieties comprising at least two functional groups selected from OH, NH₂, and COOH.

11. The targeting system according to claim 1, wherein the targeting molecule is selected from a neutral and negatively charged molecule.

12. The targeting system according to claim 6, wherein the radionuclide binding molecule (RBM) is selected from TCMC, 2,2′,2″,2′″-(1,4,7,10-tetraazacyclododecane-1,4,7, 10-tetrayl)tetraacetic acid, Hexahydro-1H-1,4,7-triazonine-1,4,7-triacetic acid, 1,4,7-Tris(phosphonomethyl)-1,4,7-triazacyclononane, ((1,4,7-triazonane-1,4,7-triyl)tris(meth-ylene))tris(phosphinic acid), N′-[5-[[4-[[5-(acetylhydroxyamino)pentyl]amino]-1,4-dioxobu-tyl]hydroxyamino]pentyl]-N-(5-aminopentyl)-N-hydroxy-butanediamide, 2, 2′, 2″, 2″′-((((car-boxymethyl)azanediyl)bis(ethane-2,1-diyl))bis(azanetriyl))tetraacetic acid, 3,12-bis(carbox-ymethyl)-6,9-dioxa-3,12-diazatetradecanedioic acid, 2,2′,2″,2″′-(ethane-1,2-diylbis(azanetriyl))tetraacetic acid, 7-[2-[bis(carboxymethyl)amino]-3-(4-nitrophenyl)propyl]hexahydro -1H-1,4,7-Triazonine-1,4(5H)-diacetic acid, 2-(4,7-bis(carboxymethyl)-1,4,7-triazonan-1-yl)pentanedioic acid, 2-(4,7, 1O-tris(carboxymethyl)-1,4,7, 10-tetraaza-cyclododecan-1-yl)-pentanedioic acid, 1,4,7-triazacydononane-1-[methyl(2-carboxy-ethyl)-phosphinic acid]-4,7-bis[methyl(2-hydroxymethyl)phosphinic acid], 3,6,9, 1 5-tetraazabicyclo[9,3, 1]pentadeca-1 (15), 11, 13-triene-3,6,9-triacetic acid, N,N″-bis[2-hy-droxy-5-(carb oxy ethyl)-benzyl]ethylenediamine-N,N″-diacetic acid, N,N′-bis(2,2-dime-thyl-2-mercaptoethyl)ethylenediamine-N,N′-diacetic acid, 1-(4-carboxymethoxybenzyl)-N-N′-bis[(2-mercapto-2,2-dimethyl)ethyl]-1,2-ethylenediamine-N,N′-diacetic acid, N,N′-di -pyridoxylethylenediamine-N,N′-diacetic acid, 1,1,1-Tris-(aminomethyl)ethane, nitrilotrime-thylphosphonic acid, 2-BAPEN, 2,2′,2″,2″′-(1,4,8,1 1-tetraazacyclotetradecane-1,4,8,11-tetrayl)tetraacetic acid, and compounds comprising one of these radionuclide binding molecules.

13. A medicament comprising the targeting system according to claim 4, wherein the medicament is for use in the treatment of a cancer, of Acute Leukemia, AML, anaplastic large cell lymphoma, neuroblastoma, bladder cancer, bone marrow, brain, breast and ovarian cancer, colorectal, urothelial carcinomas, cholangiocarcinoma, chronic lymphocytic leukaemia, non-Hodgkin lymphoma, non-Hodgkin' s disease, distant colorectal cancer, GEP-NET, glioma, glioblastoma, colorectal, lung, oesophageal and stomach cancer, head & neck carcinoma, haematological cancers, HER2 positive breast cancer, HR-Pos and HER2-negative breast cancer, immuno oncology, late-stage melanoma, leukaemia, lung & breast Cancers, lymphoma, medullary thyroid cancer, NSCLC, SCLC, melanoma, metastatic breast cancer, metastatic colorectal cancer, advanced GIST, metastatic mCR-prostate cancer (bone), metastatic melanoma, myeloma, multi cancers, myeloid leukaemia, myeloid leukaemia, Philadelphia chromosome positive acute lymphoblastic leukaemia, neuroblastoma, neuroendocrine tumours, small cell lung cancer, Non-small cell lung cancer, small cell lung cancer, non-Hodgkin lymphoma, Parkinson disease, primary Kidney Cancer, Advanced renal cancer, advanced primary liver cancer, FLT3-ITD passive AML and radioactive iodine resistant advanced thyroid carcinoma), prostate cancer, renal cell carcinoma (RCC), Imatinib-resistant GIST, Renal cell carcinoma, soft tissue sarcoma, rheumatoid arthritis, psoriatic arthritis, and ulcerative colitis, and for Targeted delivery of vinblastine, and Thyroid cancer.

14. A medicament dosage comprising an effective amount of the targeting system of claim 1 for treatment of cancers wherein cancers are selected from breast cancer, kidney cancer, non-Hodgkin's lymphoma, prostate cancer, bladder cancer, esophagus cancer, pharynx and larynx cancer, lung cancer, brain cancer, pancreas cancer, colorectal cancer, head neck cancer, glioblastoma, myeloma, myometrium, ovarian cancer, gastrointestinal stromal cancer, tumours thereof, and-metastases thereof.

15. The medicament dosage according to claim 14, comprising an amount of 0.1-1000 nMole targeting system/kg body weight and/or is provided in a physiological acceptable solution of 1-50 ml.

16. Multiple dosages for use as a medicament according to claim 14, for intermittent application, with intervals of 0.2-4 hours.

17. The targeting system according to claim 1, wherein the at least one first chemical moiety is selected from CF3, phenyl, F, and combinations thereof.

18. The targeting system according to claim 17 wherein the at least one first chemical moiety is selected from

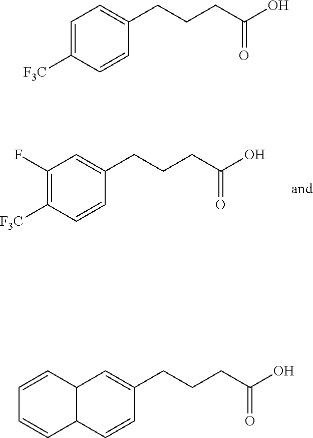 [SMILES: O=C(O)CCCc1ccc(C(F)(F)F)cc1 ; O=C(O)CCCc1ccc(C(F)(F)F)c(F)c1 ; O=C(O)CCCC1=CC2C=CC=CC2C=C1]

19. The targeting system according to claim 3, wherein further groups R5, R6, R7, and R8, are present, wherein R5, R6, R7, and R8, are selected each individually from H, and alkyl, wherein the aromatic ring systems comprise further functional groups R1, R2, and-substituents, wherein R1, R2, are selected each individually from H, sulphonate, and sulphonamide, wherein the chain of alternating single and double bonds L is interrupted by one or more partly and fully saturated ring structures, wherein the saturated ring structure further comprises functional groups R9, being selected from R10, H, AA and BB, wherein R10 is selected from, H, SO₃H, Cl, —N—C═O—(CH₂)_{q}—Y₃ (q=1-6), —(CH₂)_{r—Y}₄ (r=1-6), Y₃ and Y₄ are each independently one of H, COOH, SO₃H, and CN.
